(19) United States
(12) Patent Application Publication
(10) Pub. No.: US 2004/0236232 A1
(43) Pub. Date: Nov. 25, 2004

(54) Method and apparatus for acquiring and processing images of a tooth

(75) Inventors: Gediminas Jonusauskas, Talence (FR); Claude-Antoine Rulliere, Villenave D-Ornon (FR); Jean Oberle, Bordeaux (FR); Emmanuel Abraham, Villenave D'Ornon (FR)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Centre National De La Recherche Scientifique

(21) Appl. No.: 10/720,998

(22) Filed: Nov. 24, 2003

Related U.S. Application Data
(63) Continuation of application No. PCT/FR02/01776, filed on May 27, 2002.

(30) Foreign Application Priority Data
Jun. 1, 2001 (FR) 0107274

(51) Int. Cl. A61B 6/00

(52) U.S. Cl. 600/477

(57) ABSTRACT

A method and apparatus for acquiring and processing images of a tooth, comprising means for exciting a zone (12) of a tooth by monochromatic ultraviolet light pulses in alternation with visible light pulses, video means (32, 34, 36, 38) for taking fluorescence images emitted by the tooth in two respective high and low energy wavelength bands of the emission spectrum, and data processor means (40) for taking the ratio of the fluorescence spectrum intensities in said two wavelength bands and for deducing therefrom the presence or the absence of caries in the zone (12) under examination of the tooth.

The present invention relates to a method and apparatus for acquiring and processing images of a tooth in order to detect dental caries.

Proposals have already been made, in particular in U.S. Pat. Nos. 4,290,443 and 4,479,499, for a method of detecting dental caries in the mouth of a patient, which method consists in lighting a zone of a tooth with monochromatic light, in measuring the intensity of the luminescence emitted by the tooth at two predetermined wavelengths, at one of which wavelengths, zones of the tooth having caries and zones of the tooth not having caries have substantially the same luminance response to excitation by the lighting, and at the other of which wavelengths the intensity of the luminescence emitted is greater for a zone having caries, that method consisting finally in comparing the measurements taken at said two wavelengths for a zone of the tooth that is known not to have caries and for a zone of the tooth that is under examination.

Proposals have been made in particular to light the tooth with monochromatic light at a wavelength lying in the range about 350 nanometers (nm) to 600 nm, and to measure the intensity of the light emitted by the tooth at a first wavelength lying in the range 440 nm to 470 nm and at a second wavelength lying in the range 560 nm to 640 nm.

A drawback with that known technique is that by using lighting with monochromatic light at a wavelength lying in the range about 350 nm to 600 nm, it is not known which component of the tooth produces the luminescent response to the lighting of the tooth, thus constituting a factor of uncertainty concerning the results since, in particular, the luminescent response of the organic portion of the tooth varies as a function of a certain number of factors such as how well the teeth have been brushed, the eating habits of the patient, etc.

Another drawback of that known technique lies in measuring the integrated luminescent response of the lighted zone of the tooth at two wavelengths only, such spot measurement not giving sufficient information concerning the state of the zone being examined of the tooth, which constitutes another factor of uncertainty on the quality of the results.

Another drawback is that if the zone under examination of the tooth is lighted by monochromatic light at a wavelength shorter than about 400 nm, i.e. lying outside the visible spectrum, the practitioner does not know exactly which zone of the tooth is being lighted and might have caries. This leads in practice to restricting the use of that technique to lighting with visible light, thereby leading to results that are imprecise or erroneous in the measurements for reasons that are explained in greater detail below.

A particular object of the invention is to provide a method and apparatus of the above-specified type but not presenting the above-specified drawbacks of the known technique.

Another object of the invention is to provide a method and apparatus of this type enabling dental caries to be detected reliably and accurately, even at an early stage of development, and also making it possible to display and locate the zone under examination of the tooth accurately.

To this end, the invention provides a method of acquiring and processing images of a tooth, the method consisting in lighting a zone of a tooth in monochromatic light and in picking up the luminance emitted by the lighted zone of the tooth, the method being characterized in that it also consists in:

lighting said zone of the tooth in monochromatic light at a wavelength selected to excite emission of fluorescence by the mineral portion of the tooth;

using video means to take images of the lighted zone of the tooth in two wavelength bands, one of which is in a high energy portion and the other of which is in a low energy portion of the emission spectrum;

measuring at each point of the image the spectral intensity of the emitted fluorescence in these two wavelength bands; and

taking the ratio of the measured intensities at each point in the two above-specified wavelength bands and comparing said ratio with predetermined values.

The detection of caries, if any, is based on detecting fluorescence emitted in two wavelength bands by the mineral component of a tooth, which component is constituted by monocrystals of hydroxylapatite. Dental caries are constituted by progressive and localized demineralization of the hard tissue of the tooth surface caused by the acids produced by bacteria and leading to a reduction in the size of the hydroxylapatite crystals and by a change to the photo-physical properties of the surface of the tooth. In response to being excited by light at a suitable wavelength, the mineral component of a tooth emits fluorescence which is red shifted if the tooth is suffering from caries. By measuring the spectral intensity of the fluorescence emitted in two wavelength bands, one of which is in the high energy portion and the other in the low energy portion of emission spectrum, and by taking the ratio of these two measurements, values are obtained which are equal to about 2-3 for enamel, to about 4 for dentine, and to about 0.5-1 for caries, these values being independent of the stage of development of the caries and of the presence of coagulated organic matter.

In addition, the point-by-point image showing the ratio of spectral intensity measurements makes it possible to ignore the influence of the shape of the surface of the illuminated zone of the tooth and thus to ignore variations due to the presence of folds or indentations in the surface of the tooth, to ignore the inclination of said surface relative to the optical axis of the detection device, and to ignore non-uniformity in lighting of the zone under examination of the tooth.

Overall, the method of the invention thus enables dental caries to be detected reliably and accurately, even at an early stage of development. It also makes it possible to monitor accurately the effectiveness of surgical intervention to remove demineralized dental matter, so as to eliminate portions suffering from caries completely without spoiling healthy portions of the tooth.

According to other characteristics of the invention, the lighting wavelength lies in the range about 300 nm to 370 nm and the spectral intensity of the emitted fluorescence is measured in a wavelength band that extends between an excitation wavelength and a wavelength lying in the range about 450 nm to 600 nm, and in a wavelength band lying in the range about 550 nm-600 nm to about 750 nm-800 nm.

The sensitivity and the accuracy of the detection of dental caries are thus maximized.

According to another characteristic of the invention, the method consists in lighting said zone of the tooth with alternating pulses of light at the above-mentioned wavelengths and at a wavelength in the visible spectrum, in taking images of said zone illuminated successively at said two wavelengths using the video means, and in transmitting the images to image processor and display means.

Advantageously, the method also consists in accumulating images taken at said two wavelengths prior to processing them, and displaying an image of the fluorescence emitted by the illuminated zone of the tooth and an image of said zone illuminated in visible light.

This double display enables the practitioner to visualize and locate accurately the zone of the tooth under examination.

Advantageously, it is possible to use the same laser generator to produce the pulses for exciting fluorescence and the pulses for providing lighting with visible light, these pulses being of a duration lying in the range several microseconds to one nanosecond or less, for example, the laser generator possibly also being used to produce synchronizing pulses, e.g. in the infrared. It is possible in particular to use a laser generator of the Q-switched Nd/YAG type which produces pulses of very short duration at wavelengths of 1064 nm for synchronization, 532 nm (second harmonic) for lighting with visible light, and 355 nm (third harmonic) for exciting fluorescence.

The invention also provides apparatus for performing the above-described method, the apparatus comprising a source of monochromatic light, optical means for lighting a zone of the tooth by the light emitted by said source and for picking up light coming from the tooth, transmission means for transmitting the light that has been picked-up to spectral filter means, photodetectors sensing the light coming from the spectral filter means, and data processor means receiving the signals output by the photoreceivers, the apparatus being characterized in that the light source emits at a wavelength selected to excite the emission of fluorescence by the mineral portion of the tooth, in that it includes video means for taking images of the lighted zone of the tooth, in association with shutter or time gate means for alternately taking images of the tooth illuminated in visible light and images of the fluorescence of the tooth in high energy and low energy wavelength bands respectively of the emission spectrum, and in that the data processor means are provided to take the ratio at each point of the image between the intensities measured in said wavelength bands of the emission spectrum.

The spectral filter means used comprise, for example, interchangeable color filters, or an acousto-optical filter, or a liquid crystal filter, or a set of dichroic mirrors.

Advantageously, the transmission means comprise an optical fiber image guide or a boroscope having a glass bar with a transverse refractive index gradient.

The invention will be better understood and other characteristics, details, and advantages thereof will appear more clearly on reading the following description given by way of example and with reference to the accompanying drawings, in which:

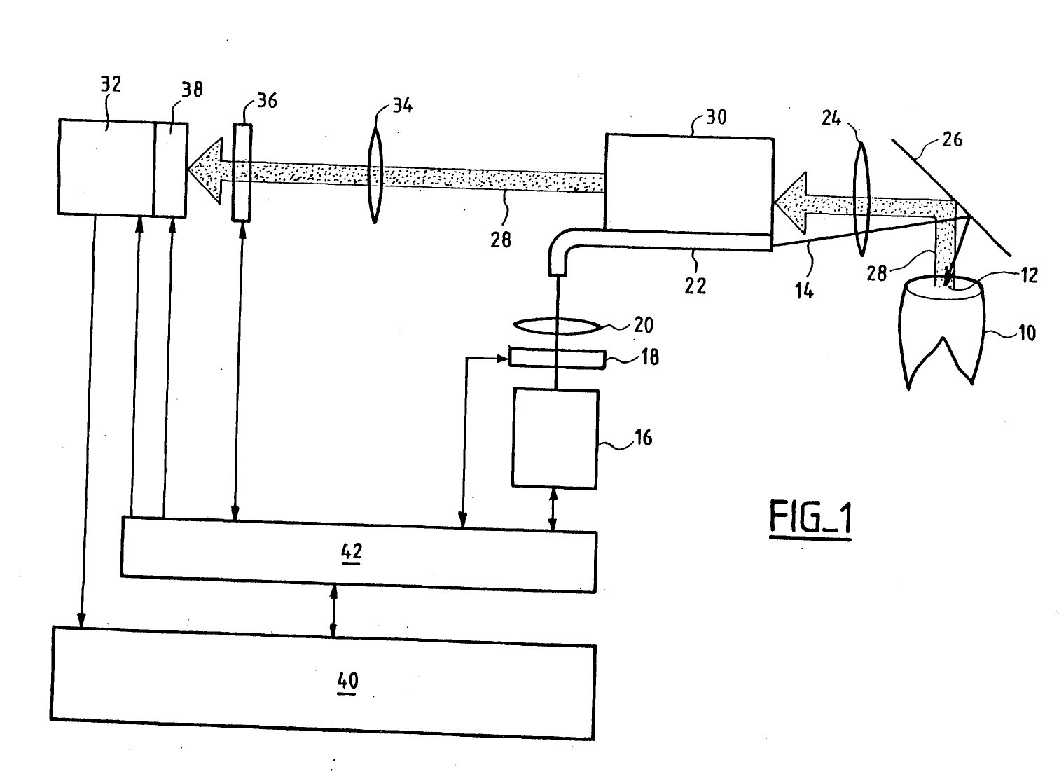

FIG. 1 is a diagram of the essential components of apparatus of the invention;

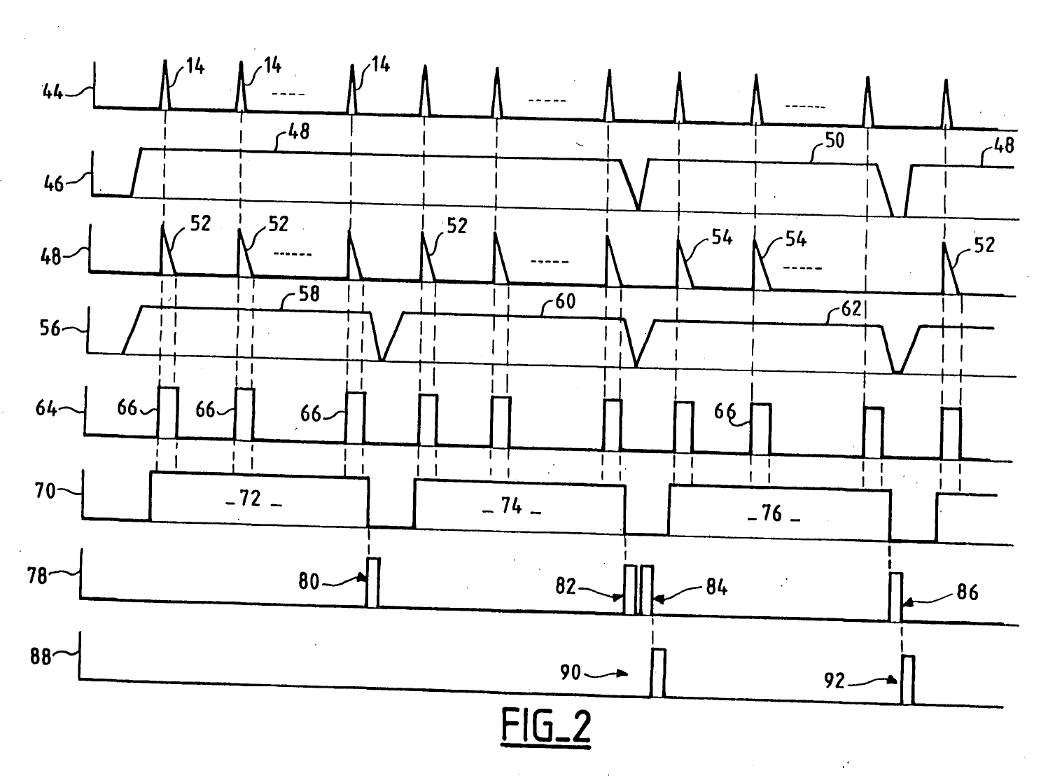

FIG. 2 shows the timing of operation of the apparatus;

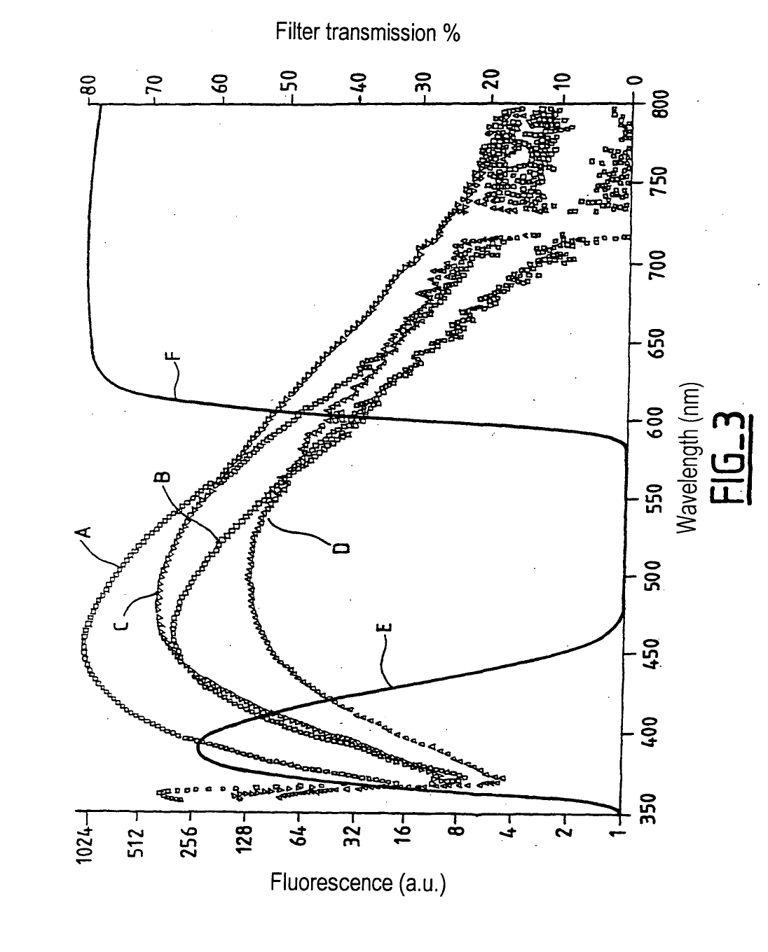

FIG. 3 is a diagram showing the fluorescence spectra of various portions of a tooth and the wavelength bands used for measuring the spectral intensity of fluorescence; and

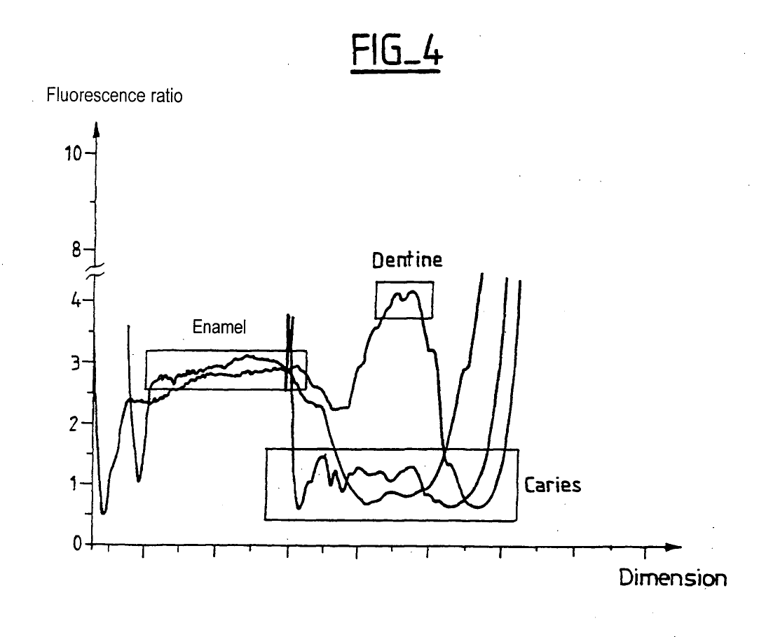

FIG. 4 is a graph showing variations in the ratios of the measured intensities of fluorescence in the two wavelength bands for different portions of a tooth.

The method and the apparatus of the invention are based on illuminating a zone 12 of a tooth 10 by a beam 14 of ultraviolet monochromatic light exciting emission of fluorescence by the mineral portion of the tooth, and in detecting images of the fluorescence of the zone 12 of the tooth in two different wavelength bands, in the high energy portion and in the low energy portion of the emission spectrum, with the ratio at each point of the spectral intensity measurements of the fluorescence in these two bands serving to determine whether the zone 12 under examination of the tooth does or does not present caries.

For a good understanding of the nature of the problem solved by the invention, it is recalled that caries is an infectious disease in which lesions are signs and symptoms that appear a long time after the primary infection and the initiation of the pathological process, where prevention has not been undertaken or has failed, the lesions being due to physicochemical phenomena whereby acids produced by the metabolism of bacterial plaque has led to surface demineralization of the calcified tissue of the tooth.

At present, detecting dental pathology relies essentially on direct visual evaluation and on tactile evaluation performed by a practitioner, or on X-ray images. The ionizing nature of X-rays means that they cannot be used repetitively and routinely for prevention of caries and for monitoring care. Furthermore, visual or tactile evaluation by a practitioner does not enable caries to be detected at any early stage of development when remineralization of the zones under attack is still possible by in situ precipitation of calcium and phosphate ions which would avoid the need for curative surgical intervention.

The apparatus of the invention serves specifically to perform such early detection, in a manner that is reliable and independent of individuals.

The apparatus of the invention shown diagrammatically in FIG. 1 comprises a laser generator 16, e.g. of the Q-switched Nd/YAG type which produces pulses at different wavelengths, e.g. 1064 nm, 532 nm, and 355 nm at a repetition frequency of 12 kilohertz (kHz), and associated both with spectral filter means 18 and with a lens 20 focusing on the inlet of an optical fiber 22 for transmitting pulses 14 which, on leaving the optical fiber 22, pass through a lens 24 and are reflected by a mirror 26 towards the zone 12 under examination of the tooth 10.

By way of example, the spectral filter means 18 comprise two interchangeable color filters, one of which transmits wavelengths at 355 nm and stops wavelengths at 532 nm, and the other of which, conversely, transmits wavelengths at 532 nm and stops wavelengths at 355 nm. These two filters are mounted on a support of electromechanical type, for example, enabling them to be placed in turn on the outlet from the laser generator 16.

The means 24, 26 for lighting the zone 12 of the tooth also form means for picking up the emitted fluorescence 28 which is focused on the input of optical transmission means 30 such as an image guide formed by a bundle of optical fibers, for example.

The means 22, 24, 26, and 30 are advantageously combined in a one-piece unit which the practitioner can hold in one hand and insert one end of the unit in the mouth of a patient to examine the teeth of the patient.

The light beam 28 leaving the transmission means 30 is directed towards video acquisition means 32 via a lens 34, spectral filter means 36, and means 38 forming a shutter or time gate.

The spectral filter means 36 comprise two color filters of bandpass type, one of which transmits wavelengths lying between the excitation wavelength and about 450 nm-600 nm, and the other of which transmits wavelengths lying in the range about 550 nm-600 nm to 750 nm-800 nm.

The means 38 forming a shutter or a time gate are controlled so as to pass to the video acquisition means 32 either wavelengths in the high energy band, or else wavelengths in the low energy band, or indeed wavelengths corresponding to the pulses at the wavelength of 532 nm which are reflected and diffused by the zones 12 under examination of the tooth. The colored filters of the means 36 are mounted on a common electromechanical type support which interposes them in turn on the optical axis of the light leaving the transmission means 30 and which puts neither filter on said axis when transmitting light corresponding to reflection and diffusion of pulses at the wavelength of 532 nm.

The means 38 forming a shutter or a time gate are formed, for example, by an image intensifier with voltage modulation on an acceleration grid, the shutter remaining open only to pass pulses of fluorescence and of visible light coming from the tooth 10. When the shutter is closed, it prevents all light that does not carry information about the properties of the surface of the tooth from passing. In a variant, it is also possible to use a mechanical or a liquid crystal shutter, an acousto-optical deflector, a camera having a very short duration of charge accumulation, etc.

The image acquisition means 32 are preferably formed by a black and white matrix camera having charged-coupled device (CCD) type photoreceivers, with the output thereof being connected to the input of data processor means 40, such as a microcomputer of personal computer (PC) type or the like. Synchronizing means 42 are associated with the data processor means 40, with the generator 16, with the filter means 18 and 36, with the shutter means 38, and with the video acquisition means 32. These synchronizing means 42 receive the synchronizing pulse produced at the wavelength of 1064 nm by the laser generator 16.

The apparatus is used as follows:

the means 22, 24, 26, and 30 form a probe which the practitioner can hold and point towards the zone 12 for examination on the tooth 10. The pulses emitted by the laser generator at the wavelengths of 532 nm and 355 nm are transmitted in alternation by the spectral filter means 18 and the optical fiber 22 to the zone 12 of the tooth. The pulses at 355 nm are absorbed by components of tissue at the surface of the tooth, which components become de-excited by emitting fluorescence during a very short duration, typically a few nanoseconds. Similarly, the visible light pulses at a wavelength of 532 nm are reflected and diffused by the surface of the tooth. The light pulses coming from the tooth are picked up by the optical means 24, 26 and transmitted by the means 30 to the spectral filter means 36 associated with the video acquisition means 32 by the means 38 forming a shutter or a time gate. The video images acquired by the means 32 are transmitted to the data processor means 40 and are displayed on suitable means, in particular a display screen.

The principal steps of the method are shown diagrammatically in FIG. 2, in which: 44 shows the emission of light pulses by the generator 16; 46 shows the spectral filtering of these pulses by the means 18 which enable pulses for exciting fluorescence to be transmitted during a first period 48, followed by pulses of visible light during a second period 50, and so on; 48 shows the emission of pulses of fluorescence 52 by the zone 12 under examination of the tooth, followed by pulses 54 of visible light which are reflected and/or diffused by the surface of said zone; and 56 shows the spectral filtering of the light pulses transmitted by the means 30, this spectral filtering being performed in succession in a high energy band 58 and in a low energy band 60, and finally, at 62, by allowing visible light pulses as reflected and/or diffused by the surface of the tooth to pass.

Thereafter, at 64, there can be seen the acquisition of fluorescence images in the high and low energy bands of the emission spectrum and visible light images during the intervals 66 when the shutter or time gate means 38 are open.

This leads to 70 which shows high energy band fluorescence images being accumulated at 72, low energy band fluorescence images being accumulated at 74, and visible light images being accumulated at 76.

Thereafter, the processing performed by the means 40 comprises, at 78, storing high energy band fluorescence images 80 and storing low energy band fluorescence images 82, and also performing processing 84 on the fluorescence images and storing visible light images at 86, followed at 88 by displaying 90 images that result from fluorescence and images 92 that result from visible light. The stages of operation of the apparatus may be interchanged.

In more detailed manner, the processing of the fluorescence images that is performed at 84 consists in measuring the spectral intensity of the fluorescence emitted in the above-mentioned high and low energy bands, in taking the ratio thereof, and in comparing the ratio with predetermined values.

FIG. 3 is a diagram showing curves plotting variation in the fluorescence emitted as a function of wavelength by dentine (curve A), by enamel (curve B), by caries at an early stage of development (curve C), and by caries at an advanced stage of development (curve D).

Curves E and F show the passbands of the high energy and low energy filters of the spectral filter means 36.

It can be seen that the fluorescence curves are offset towards the red for caries and that the emitted fluorescence intensity is lower from advanced caries because of the presence of coagulated organic matter.

The treatment performed on the fluorescence images in the passbands E and F consists in measuring the intensity of the fluorescence energy in these two bands and in taking the ratio thereof. Three examples of variations in this ratio are shown diagrammatically in FIG. 4 as a function of a spatial dimension plotted along the abscissa and measured over a tooth. It can be seen in particular that the ratio of fluorescence energy in the high energy band divided by fluorescence energy in the low energy band of the emission spectrum can vary between values lying in the range about 2 to 3 for enamel, which are substantially equal to 4 for dentine, and which lie in the range 0.5 to 1 for portions suffering from caries.

The ratio of these intensities in the fluorescence images makes it possible to ignore the shape of the surface under examination of the tooth, i.e. the presence of folds or indentations, and also to ignore the inclination of said surface relative to the optical axis of illumination and non-uniformity of illumination.

Displaying fluorescence images and visible light images on a display screen allows the practitioner to locate accurately a zone of the tooth that is suffering from caries. It is also possible to show variations in the fluorescence energy ratios in false colors, so that zones suffering from caries appear to be red, for example, and are clearly visible to the practitioner.

Naturally, various modifications may be made to the means described and shown: for example, it is possible to use other laser generators, e.g. having crystal or glass doped with Nd³⁺, Yb, etc., using harmonic generation, or nitrogen lasers operating at 337 nm, excimer lasers operating at 308 nm or at 351 nm, semiconductor lasers, electrical discharge ultraviolet lamps, etc. In addition, the image transmission means 30 which comprise a flexible image guide having a diameter of 1 millimeter and a length of about 1 meter for example and possibly containing 30,000 individual optical fibers in a preferred embodiment of the invention may be replaced by a system of mirrors and lens or by a boroscope based on using a bar of glass with a transverse refractive index gradient.

The spectral filter means may be constituted by an acousto-optical filter, a set of dichroic mirrors, a liquid crystal filter, etc.

The video acquisition means 32 which are formed by a matrix of CCD sensors in the preferred embodiment of the invention may be replaced by matrices of photodiodes, vidicons, CMOS sensors, and they may have a video output that is analog or digital, monochrome, or in color.

Naturally, the means 22 for transmitting the light for lighting may comprise a plurality of optical fibers which are arranged at their ends to enable the laser beam produced by the generator 16 to be injected efficiently with uniform intensity, and at the other end to light the zone 12 of the tooth uniformly.

It is also possible to use optical means 24, 26 for lighting and pickup purposes that are different from those described and shown.

1. A method of acquiring and processing images of a tooth, the method consisting in lighting a zone (12) of a tooth in monochromatic light and in picking up the luminance emitted by the lighted zone of the tooth, the method being characterized in that it also consists in:
lighting said zone (12) of the tooth in monochromatic light at a wavelength selected to excite emission of fluorescence by the mineral portion of the tooth;
using video means (32) to take images of the lighted zone of the tooth in two wavelength bands, one of which is in a high energy portion and the other of which is in a low energy portion of the emission spectrum;
measuring the spectral intensity of the emitted fluorescence in these two wavelength bands at each point of said images; and
taking the ratio at each point of the measurements in the two above-specified wavelength bands and comparing said ratio with predetermined values.

2. A method according to claim 1, characterized in that the lighting wavelength lies in the range about 300 nm to 370 nm.

3. A method according to claim 1 or claim 2, characterized in that the wavelengths of the above-specified bands lie in the range excitation wavelength to about 450nm-600 nm, and in the range about 550 nm-600 nm and about 750 nm-800 nm, respectively.

4. A method according to any preceding claim, characterized in that it consists in lighting said zone (12) of the tooth by alternating pulses at two different wavelengths, one in the ultraviolet and the other visible, in using the video means (32) to take fluorescence images in said high and low energy bands of the zone lighted by pulses of ultraviolet wavelength, and images of said zone (12) lighted by pulses of visible wavelengths, and in transmitting these images to data processor and display means (40).

5. A method according to claim 4, characterized in that it consists in accumulating fluorescence images in the above-specified high and low energy bands and visible wavelength images prior to processing them and displaying an image of the fluorescence spectral intensity ratio and an image of said zone (12) of the tooth lighted in visible light.

6. A method according to any preceding claim, characterized in that it consists in using the same laser generator (16) to produce fluorescence exciting pulses (14) and lighting at a visible wavelength, said pulses being of a duration lying in the range a few microseconds to one nanosecond or less.

7. A method according to claim 6, characterized in that it consists in using the same laser generator (16) to produce synchronizing pulses, e.g. in the infrared.

8. Apparatus for performing the method described in any preceding claim, the apparatus comprising a source (16) of monochromatic light, optical means (22, 24, 26) for lighting a zone (12) of the tooth, and for picking up light coming from the tooth, means (30) for transmitting the picked-up light to spectral filter means (36), photoreceivers sensing the light leaving the spectral filter means (36), and data processor means (40) receiving the signals delivered by the photoreceivers, the apparatus being characterized in that the source (16) emits at a wavelength selected to excite emission of fluorescence by the mineral portion of the tooth, in that it comprises video means (32) for taking images of the lighted zone (12) of the tooth, associated with shutter or time gate means (38) for taking alternately images of the zone (12) of the tooth as illuminated in visible light and fluorescence images of said zone (12) in high energy and low energy wavelength bands respectively of the emission spectrum, and in that the data processor means (40) are designed to take the ratio at each point of the image between the intensities measured in said wavelength bands of the emission spectrum.

9. Apparatus according to claim 8, characterized in that the spectral filter means (36) comprise interchangeable color filters or an acousto-optical filter or a liquid crystal filter or a set of dichroic mirrors.

10. Apparatus according to claim 8 or claim 9, characterized in that the transmission means (30) comprise an optical fiber image guide or a glass bar boroscope having a transverse refractive index gradient.

11. Apparatus according to any one of claims 8 to 10, characterized in that the lighting means comprise a laser generator (16) associated with spectral filter means (18) and controlled to produce pulses at different wavelengths for lighting the tooth in ultraviolet light and in visible light.

12. Apparatus according to claim 11, characterized in that the laser generator (16) is also controlled to produce synchronizing pulses, e.g. in the infrared.

13. Apparatus according to any one of claims 8 to 12, characterized in that it further comprises synchronizing means (42) connected to the light source (16), to the video means (32) for taking images, to the spectral filter means (18, 36), to the shutter or time gate means (36), and to the data processor means (40).
